(19) United States
(12) Patent Application Publication
(10) Pub. No.: US 2018/0084996 A1
(43) Pub. Date: Mar. 29, 2018

(54) A WIRELESS IMAGING APPARATUS AND RELATED METHODS

(71) Applicants: Wei SU, Sunnyvale, CA (US); VISUNEX MEDICAL SYSTEMS CO. LTD., Grand Cayman (KY)

(72) Inventors: Wei SU, Sunnyvale, CA (US); Li XU, San Ramone, CA (US)

(73) Assignee: VISUNEX MEDICAL SYSTEMS CO. LTD., Grand Cayman (KY)

(21) Appl. No.: 15/563,388

(22) PCT Filed: Mar. 31, 2016
(86) PCT No.: PCT/US16/25251

§ 371 Date: Sep. 29, 2017

Related U.S. Application Data
(60) Provisional application No. 62/141,209, filed on Mar. 31, 2015.

(51) Int. Cl. A61B 3/14; A61B 3/00; A61B 5/00

(52) U.S. Cl. CPC A61B 3/14; A61B 3/0041; A61B 5/6898; A61B 2560/0431; A61B 5/0013; A61B 3/0025; A61B 3/0033

(57) ABSTRACT

Various embodiments of a wireless imaging apparatus are disclosed. The wireless imaging apparatus can comprise an optical imaging system, a light source and a modified mobile device. In some embodiments, the optical imaging system can be positioned outside the modified mobile device. In some embodiments, the wireless imaging apparatus can further comprise a microcontroller, and at least one of an input port, an output port, a control button, and an output signal of the modified mobile device can be connected to the microcontroller and configured to control an image capturing process. Various embodiments disclose a method to control an imaging process of a wireless imaging apparatus comprising a light source, an optical imaging system, a modified mobile device and a microcontroller.

### CROSS REFERENCE TO RELATED APPLICATIONS

This application claims the benefit of U.S. Application No. 62/141,209, titled: “A WIRELESS IMAGING APPARATUS AND RELATED METHODS”, filed Mar. 31, 2015, which is incorporated herein by reference.

### INCORPORATION BY REFERENCE

The following U.S. patent applications are herein incorporated by reference in their entirety: U.S. application Ser. No. 14/220,005, titled “Eye Imaging Apparatus and Systems” filed on Mar. 19, 2014, which is a continuation-in-part of U.S. application Ser. No. 13/757,798, titled “Portable Eye Imaging Apparatus” filed Feb. 3, 2013 which claims the benefit of: U.S. Provisional Application No. 61/593,865 filed Feb. 2, 2012, U.S. application Ser. No. 14/312,590, titled “Mechanical Features of an Eye Imaging Apparatus” filed on Jun. 23, 2014, and U.S. application Ser. No. 14/191,291, titled “Eye Imaging Apparatus with a Wide Field of View and Related Methods” filed on Feb. 26, 2014, which is a continuation-in-part of U.S. application Ser. No. 13/845,069, titled “Imaging and Lighting Optics of a Contact Eye Camera” filed Mar. 17, 2013, which claims the benefit of U.S. Provisional Application No. 61/612,306 filed Mar. 17, 2012.

All publications and patent applications mentioned in this specification are herein incorporated by reference in their entirety to the same extent as if each individual publication or patent application was specifically and individually indicated to be incorporated by reference.

### FIELD

Various embodiments of the disclosure relate generally to a wireless imaging apparatus and related methods, and particularly, a wireless imaging apparatus comprising a modified mobile device and related methods.

### BACKGROUND

Imaging apparatuses have become increasingly important in many applications. For example, an eye imaging apparatuses has been widely used in medical procedures instead of a conventional viewing instrument such as an ophthalmoscope. Imaging apparatuses have the advantages of being able to record the images, and enable the physicians to compare different images for diagnostic and treatment purposes.

However, there is a need for an imaging apparatus with wireless communication capability. For example, the patients in remote areas may not have convenient access to medical facilities. A wireless imaging apparatus is needed to transmit the images of the patients to the physicians in hospitals and medical facilities for timely diagnosis and treatment.

It can be expensive to develop an imaging apparatus with high speed wireless transmission capability from the very beginning. There have been some efforts to combine an imaging apparatus or a view instrument with a conventional mobile device such as a smart phone by using an adapter. For example, U.S. application Ser. No. 13/525,598 titled “Smart-phone Adapter for Ophthalmoscope” proposed an adapter which connects a camera of a smart phone to an ophthalmoscope at multiple locations. However, it is very difficult to achieve precise optical alignment of the viewing instrument and the mobile device. Thus, it is difficult to obtain high quality images by simply using adaptors. In addition, many medical imaging applications may require complicated optical illumination systems. The flash light built in the mobile device may not be able to provide adequate illumination, and a light source disposed outside the mobile device may be needed for such image applications. Furthermore, in order to obtain high quality optical images, the camera and the light source may need to be controlled and synchronized. A conventional mobile device may not be capable of providing control and synchronization of the device disposed outside, such as the light source. Therefore, there is a need to develop a wireless imaging apparatus that is capable of providing high quality images with high speed wireless communication capability for medical and other applications.

### SUMMARY OF THE DISCLOSURE

The present disclosure relates to various embodiments of a wireless imaging apparatus comprising a light source, an optical imaging system and a modified mobile device. In general, the wireless imaging apparatus can be configured to utilize the high speed wireless communication capability and high computing power of the modified mobile device. The modified mobile device may be based on a modification of a mobile device.

A mobile device is defined herein as a portable device with wireless communication capability, imaging capability and a display. For example, the mobile device can comprise a wireless transmitter and a wireless receiver. The mobile device can be configured to communicate wirelessly through cellular network. The mobile device can comprise modules for wireless connectivity such as Wi-Fi, Bluetooth, and/or 3G/4G, etc. The mobile device can comprise a processor and a display, for example, a low power central processing unit (CPU) and a touch screen display. The mobile device can further comprise a lens and an image sensor. For example, the mobile device can comprise a miniature camera. The miniature camera can comprise the lens and the image sensor. The mobile device can further comprise a graphic processing unit (GPU), an operating system (such as Android or iOS mobile operating systems), input/output ports, etc.

Various embodiments described herein disclose a wireless imaging apparatus. In general, the wireless imaging apparatus can comprise a housing, a light source supported by the housing and configured to illuminate an object and a modified mobile device adapted from a mobile device. The modified mobile device can be supported by the housing and comprise a wireless transmitter and a wireless receiver, a processor configured to control an optical imaging system, and a display configured to display an image of the object. The wireless imaging apparatus can comprise an optical imaging system disposed within the housing and outside the modified mobile device. The optical imaging system can comprise a lens configured to form the image of the object, and an image sensor configured to receive the image. The wireless imaging apparatus can further comprise a cable operatively connected to the optical imaging system and the modified mobile device.

For example, the wireless imaging apparatus can comprise a modified smart phone. In some embodiments, the lens can comprise a lens of the mobile device being repositioned to be outside of the modified mobile device; the image sensor can comprise an image sensor of the mobile device being repositioned to be outside of the modified mobile device. In some embodiments, the cable has a length between 5 mm and 15 mm. For example, the cable comprises a Transmission-Line-Interconnect-Structure cable.

In some embodiments, the wireless imaging apparatus further comprises an actuator of the lens disposed outside the modified mobile device, wherein the processor is configured to control the actuator of the lens and the imaging sensor. In some embodiments, the wireless imaging apparatus further comprises a second cable operatively connected to the light source and the modified mobile device, wherein the processor is further configured to control the light source disposed outside the modified mobile device.

In some embodiments, the wireless imaging apparatus further comprises a multi-functional button disposed on the housing, wherein the multi-functional button comprises electrical switches operatively connected to the light source, the lens and the imaging sensor.

In some embodiments, the wireless imaging apparatus further comprises a microcontroller disposed outside the modified mobile device and operatively connected to the modified mobile device, the light source and the imaging sensor. For example, the cable has a second branch operatively connected to the microcontroller.

Various embodiments described herein disclose a wireless imaging apparatus. The wireless imaging apparatus can comprise a housing, a light source supported by the housing and configured to illuminate an object, and an optical imaging system disposed within the housing. The optical imaging system can comprise a lens configured to form an image of the object, and an image sensor configured to receive the image. The wireless imaging apparatus can comprise a modified mobile device adapted from a mobile device. The modified mobile device can be supported by the housing and comprise a wireless transmitter and a wireless receiver, a processor configured to control the optical imaging system, and a display configured to display the image; At least one of an input port, an output port, a control button, an input signal and an output signal of the modified mobile device can be connected to a microcontroller. The microcontroller can be operatively connected to the light source and the optical imaging system.

In some embodiments, the optical imaging system can be disposed outside the modified mobile device and connected to the modified mobile device by a cable. In some embodiments, at least one of the input port, the output port, the control button, the input signal and the output signal of the modified mobile device is connected to one of the light source and the optical imaging system. In some embodiments, the wireless imaging apparatus further comprises an independent driver to drive the light source, wherein the microcontroller is further configured to control the light source. In some embodiments, the wireless imaging apparatus further comprises a multi-functional button disposed on the housing, the multi-functional button comprising electrical switches operatively connected to the light source, the optical imaging system and the microcontroller.

In some embodiments, an audio input port of the modified mobile device is used to receive a command signal. The wireless imaging apparatus is configured to encode the command signal in the frequency of an audio signal to input into the audio port. In some embodiments, an audio output port of the modified mobile device is used to transmit a command signal. The wireless imaging apparatus is further configured to decode the command signal from an audio signal from the audio port.

In general, the modification of the conventional mobile device can include the modification of a hardware structure. For example, an input/output port of the modified mobile device can be modified to be connected to the microcontroller. The modification of the conventional mobile device can further include modification of a non-transitory, computer-readable storage medium storing a set of instructions in the modified mobile device. The instructions, when executed by a processor of the modified mobile device, can be modified to cause the processor to control the image capturing process. In some embodiments, the input/output port of the conventional mobile device can be modified to control the imaging capturing process by modification of the instructions in the non-transitory, computer-readable storage medium of the modified mobile device. In some other embodiments, a control button (e.g., a volume up button or a volume down button), and/or an output signal (e.g., a flash signal or a vibration signal) of the modified mobile device can be modified to control the image capturing process by modification of the instructions related to the control button and/or the output signal, in addition to connecting the control button and/or the output signal to the microcontroller.

In general, in order to control the image capturing process including control and synchronization of the light source and the miniature camera, the modified mobile device can include, but is not limited to, the modification of a structure of the mobile device, the modification of instructions stored in a non-transitory, computer-readable storage medium of the mobile device, and any combination thereof.

In some embodiments, the wireless imaging apparatus can comprise an independent driver to drive the light source. The independent driver can be configured to drive a more powerful light source than the conventional light source in typical mobile devices. In addition, the driver can be configured to drive multiple light sources at the same time. The microcontroller can be configured to control the light source driver.

In some embodiments, the imaging apparatus can further comprise a multi-functional button on the housing of the image apparatus. The microcontroller can be connected with the multi-functional button, which is configured to control the light source and the miniature camera. After the user pushes the multi-functional button, the microcontroller can be configured to receive a first signal in response to the pushing action, and send a second electrical signal to the modified mobile device in response to the first signal.

In some embodiments, an output port of the modified mobile device can be configured to convert a command signal to a data format recognizable by the output port. In some embodiments, an input port of the modified mobile device can be configured to recover a command signal from a signal in a data format recognizable by the input port. For example, a microphone port of the modified mobile device can be modified in order for the microcontroller and the modified mobile device to communicate a command signal other than the audio signal. The microphone/speaker port can be modified to transmit the command signal by encoding the command signal into the audio signal, and recovering the command signal by decoding the audio signal. The encoding of the command signal and decoding of the audio signal may employ a variety of conversion algorithms.

In some embodiments, a control button of the modified mobile device can be connected to the microcontroller. The control button can comprise an electrical switch configured to respond to a signal from the microcontroller. When a user pushes in the multi-functional button, the multi-functional button can be configured to send the microcontroller a trigger signal, which is a first signal, in response to the pushing action. The microcontroller can send a second signal to the control button of the modified device, in response to the first signal. The control button can send a third signal to the processor of the modified mobile device, in response to the second signal. The control button can inform the modified mobile device to control the miniature camera by starting the instructions stored in the non-transitory medium for image capturing.

In some embodiments, an output signal of the modified mobile device such as a flash signal or a vibration signal can be modified to be connected to the microcontroller. The precise synchronization of the light source with the shutter of the image sensor can be particularly important under sequential illumination. The output signal of the modified mobile device can be modified to achieve the precise synchronization. In some other embodiments, the output signal can be configured to be a handshake signal to increase the efficiency and speed of communication.

In some embodiments, the wireless imaging apparatus can comprise a user interface. The user interface can allow the user to perform precise alignment, and adjust the focus and light intensity.

Various embodiments disclose a method to control an imaging process of a wireless imaging apparatus. The imaging apparatus can comprise a light source, a miniature camera, a modified mobile device, and a microcontroller. The method can comprise allowing a user to push a multi-functional button on the wireless imaging apparatus and allow the multifunctional button to send a first signal to the microcontroller in response to the pushing action. The method can comprise allowing the microcontroller to send a second signal to at least one of an input port and a control button of the modified mobile device in response to the first signal. The method can further comprise allowing the modified mobile device to control the imaging process in response to the second signal.

Various embodiments disclose a wireless imaging system. The wireless imaging system can comprise a wireless imaging apparatus comprising a light source, a miniature camera, and a modified mobile device inside the housing configured to provide wireless communication and control an imaging capturing process. In some embodiments, the imaging apparatus can further comprise a microcontroller. The wireless imaging system can comprise a base station. The base station can comprise a control panel, a computing module, a display, and a communication module. The wireless imaging apparatus can be configured to communicate with the base station wirelessly. In some embodiments, the base station can further comprise a foot switch configured to communicate with the wireless imaging apparatus wirelessly.

### BRIEF DESCRIPTION OF THE DRAWINGS

The novel features of the disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view that schematically illustrates a wireless imaging apparatus according to various embodiments of the disclosure.

FIG. 2A is a perspective view that schematically illustrates a wireless imaging apparatus comprising a removable front imaging module and a main module according to some embodiments.

FIG. 2B is a side view that schematically illustrates an integrated wireless imaging apparatus comprising a removable front imaging module and a main module according to some embodiments.

FIG. 3A is a schematic of an example of an optical system of a wireless imaging apparatus in eye imaging application according to various embodiments.

FIG. 3B is a perspective view that schematically illustrates a wireless imaging apparatus with a multi-functional control button.

FIG. 3C is a block diagram of an electronic system of a wireless imaging apparatus, for example, in an eye imaging application, according to some embodiments.

FIG. 3D is a screen shot of a user interface of a wireless imaging apparatus 300 in an eye imaging application, according to some embodiments.

FIG. 3E is a flow diagram of an example of a wireless imaging apparatus in eye imaging application, according to some embodiments.

FIG. 4A is a perspective view that schematically illustrates a wireless imaging apparatus and a base station which is a carrying case, according to some embodiments.

FIG. 4B is a perspective view that schematically illustrates a wireless imaging apparatus and a base station comprising a charging station.

FIG. 4C is a side view that schematically illustrates a wireless imaging apparatus and a base station with a foot switch.

FIG. 4D is a block diagram that schematically illustrates a wireless imaging system comprising the wireless imaging apparatus and the base station.

### DETAILED DESCRIPTION

Various aspects of the present disclosure now will be described in detail with reference to the accompanying figures. These aspects of the disclosure may be embodied in many different forms and should not be construed as limited to the exemplary embodiments discussed herein.

A mobile device is defined herein as a portable device with wireless communication capability, imaging capability and a display. For example, the mobile device can comprise a wireless transmitter and a wireless receiver. The mobile device can be configured to communicate wirelessly through cellular network. The mobile device can comprise modules for wireless connectivity such as Wi-Fi, Bluetooth, and/or 3G/4G, etc. The mobile device can comprise a processor and a display, for example, a low power central processing unit (CPU) and a touch screen display. The mobile device can further comprise a lens and an image sensor. For example, the mobile device can comprise a miniature camera comprising the lens and the image sensor. The mobile device can further comprise a graphic processing unit (GPU), an operating system (such as Android or iOS mobile operating systems), input/output ports, etc.

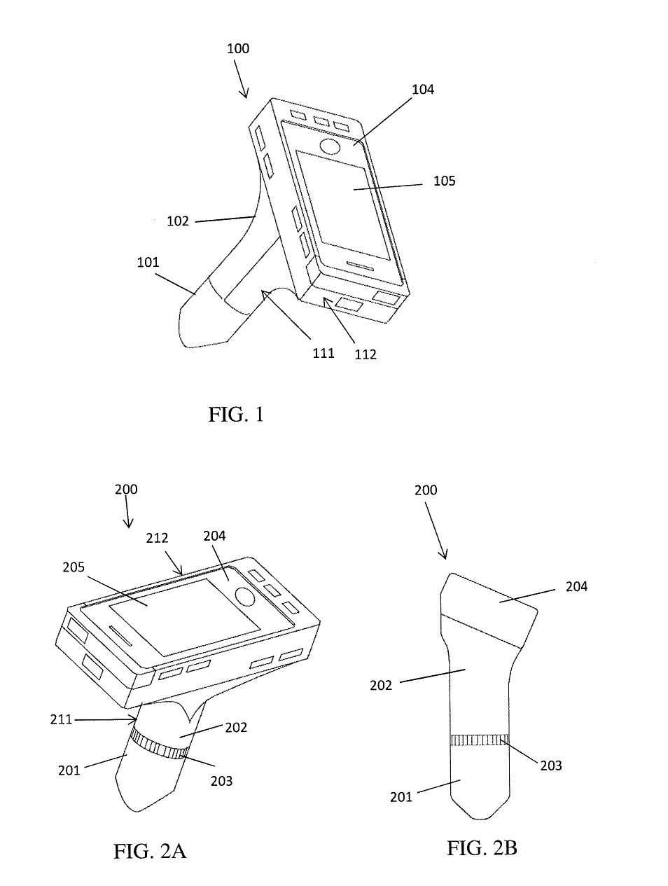

FIG. 1 schematically illustrates a wireless imaging apparatus 100 comprising a modified mobile device 304 according to various embodiments. The wireless imaging apparatus 100 can be an integration of an optical imaging apparatus with a modified mobile device 104. The modified mobile device 104 can be a modification of a mobile device. A mobile device can be a small computing device. Typically, the mobile device can be small enough to be handheld with a touch screen display. The mobile device can include, but is not limited to: smart phones, tablet computers, PCs, personal digital assistant (PDA), enterprise digital assistants, machine-to-machine (M2M) communications, industrial electronics, automotive, and medical technologies. The mobile devices can provide the computing power combined with high speed wireless communication capability.

The modified mobile device 104 can further expand the capability and flexibility of a conventional mobile device. The modified mobile device 104 may comprise a low power central processing unit (CPU), a graphic processing unit (GPU), an operating system, a touch screen display, a microphone, a speaker and a miniature digital camera, as well as other modules for wireless connectivity such as Wi-Fi, Bluetooth, and/or 3G/4G, etc. The modified mobile device 104 can be capable of providing communication through a wireless connection with digital wireless data communication networks. The modified mobile device 104 may have enhanced and expanded high speed data communication capability and a higher computing power than a conventional mobile phone. In some embodiments, for example, the modified mobile device 104 (e.g., a modified smart phone) may be based on smart phones with Android or iOS mobile operating systems, as well as other operating systems. The modified mobile device 104 may have built-in high speed data communication capability and high computing power. Modifying a conventional mobile device for imaging application may be more cost effective than designing a computing and communication unit from scratch. In addition, a touch screen display 105 of the mobile device 104 may be used as a display to review the image and may also act as a user input interface to control the image capturing process. Captured images may be transferred to other computing devices or internet-based devices, like storage units, and through wired or wireless communication systems. In various embodiments, the imaging apparatus 100 can be powered by a battery, thus improving the maneuverability and operation by the user.

The wireless imaging apparatus 100 may be used as a disease screening or medical diagnostic device for various applications. The apparatus 100 may be used in remote, rural areas where traveling to medical facilities may be inconvenient. For example, the wireless imaging apparatus 100 may be used as a portable medical imaging device for the medical applications such as eye examination, ear-nose-and-throat (ENT) examination, dermatology applications, etc. Moreover, the imaging apparatus 100 may have applications in areas other than medical applications, for example, for security screening applications in which the images from the posterior/anterior segment of the eye may be used for personal identification purposes. The imaging apparatus 100 may also be used to image animals. For example, the imaging apparatus 100 may be used to image or photograph the eyes of animals such as livestock, pets, and laboratory test animals, including horses, cats, dogs, rabbits, rats, guinea pigs, mice, etc. The wireless imaging apparatus 100 can be used in remote inspections and studies as well.

In some embodiments, for example, the imaging apparatus 100 can comprise a housing comprising a first portion 111 and a second portion 112. The first portion 111 can comprise an Imaging Capturing Unit (ICU) including an optical imaging system and an optical illumination system. The second portion 112 can comprise the modified mobile device 104. In some embodiments, the first portion ICU 111 of the imaging apparatus 100 can be cylindrical and the second portion 112 can be cuboid. For example, the cuboid portion 112 can be mounted on top of the cylindrical portion 111. The cylindrical portion 111 can have a length between about 50 mm and about 200 mm, and a diameter between about 20 mm and about 80 mm. The cuboid portion 112 may comprise a touch screen display 105. The dimension of the cuboid portion 112 can be between about 50 mm×100 mm and about 130 mm×200 mm. The cuboid portion 112 may be mounted at an angle with the cylindrical portion 111. The angle may be between about 0 and 90 degrees. The cuboid portion 112 may be perpendicular to the cylindrical portion 111 in some embodiments. The cuboid portion 112 may also be parallel to the cylindrical portion 111 in some other embodiments. The cuboid portion 112 and the cylindrical portion 111 may be integrally formed, e.g., so as to form a unitary body. For example, the cuboid portion 112 may be along a sidewall of the cylindrical portion 111, in some embodiments. In some other embodiments, the first portion 111 can be conical or any other shapes. In some other embodiments, the second portion 112 can be any other shapes, not limited to cuboid shape. The housing of the imaging apparatus 100 may be in other shapes, not limited to the combination of a cylindrical portion and a cuboid portion.

The wireless imaging apparatus 100 may be compacted to improve mobility, maneuverability, and/or portability. For example, in various embodiments, the imaging apparatus 100 can have a size less than about 250 mm along the longest dimension thereof. For example, in some embodiments the eye imaging apparatus 100 may be about 250 mm, 200 mm, 150 mm, or 100 mm along the longest dimension. In some embodiments, the eye imaging apparatus 100 may weigh less than about 2 kg. For example, the eye imaging apparatus 100 may weigh between about 0.5 kg and about 2 kg, between about 0.3 kg and about 2 kg, or between about 0.2 kg and about 2 kg in various embodiments. Advantageously, the relatively small size and weight of the imaging apparatus 100 can improve the portability of the apparatus 100 relative to other systems, thereby enabling the user to easily move the apparatus 100 to different locations and to easily manipulate the apparatus 100 during use.

In some embodiments, the wireless imaging apparatus 100 can comprise a front imaging module 101 and a main module 102. The front imaging module 101 can be configured to be repeatedly attached to and removed from the main module 102. The front imaging module 101 may be disposed at the front portion of the first portion 111 of the housing. The main module 102 may be disposed at the back port of the first portion 111 and the cuboid portion 112 of the housing. The front imaging module 101 may be removable and replaced with other imaging and illumination optics in various embodiments. When imaging and illumination optics are capable of being removed or replaced, the potential applications of the wireless imaging apparatus 100 may be significantly expanded. For example, in eye imaging application, the imaging apparatus 100, may be used to image the posterior segment of the eye with various magnifications, and under different illumination conditions, including illumination from broadband and/or narrowband light sources. The iris of the patient may or may not need to be dilated with special drugs prior to the imaging procedure. Color images from the posterior segment of the eye may also be obtained in the form of mono (2D) or stereoscopic (3D) images. The front imaging module 101 may also be designed to image the anterior segment of the eye. The front imaging module 101 may be replaced with an ultrasound probe as well.

The main module 102 can comprise a modified mobile device 104 in various embodiments. In some embodiments, for example, the modified mobile device 104 as shown in FIG. 1 can be a modified smart phone. In some other embodiments, the modified mobile device 104 may be any other suitable modified mobile device, such as a modified tablet computer, laptop computer, PDA, etc.

In some embodiments, the modified mobile device 104 can be encapsulated within the main module 102 with the touch screen monitor 105. The modified mobile device 104 may be mounted on top of the main module 102. The front imaging module 101 can be mounted on an opposite side. In some embodiments, the modified mobile device 104 can be mounted at an inclined angle, allowing easier operation of the modified mobile device 104 by the user. In some alternative embodiments, the modified mobile device 104 may also be mounted perpendicular to the optical axis of the front imaging module. The touch screen monitor 105 may be configured to display the images, including simple mono images and/or stereoscopic (3D) images. In addition, the touch screen monitor 105 may also have a touch screen control feature to enable the user to interact with the monitor 105.

The wireless imaging apparatus 100 can be designed to be operated by users with little training. In some embodiments, the first portion 111 may be usable as a handle to allow the users to easily hold the apparatus 100 with only one hand. The users may precisely adjust the position and/or angle of the apparatus with one hand, freeing another hand to work on other tasks. The second portion 112 may comprise a display and/or user input interface such as a touch screen display 105 to allow the users to navigate through the multiple functions of the imaging apparatus and control the image capturing process.

FIG. 2A and FIG. 2B schematically illustrate a wireless imaging apparatus 200 with a removable front imaging module. Unless otherwise noted, reference numerals used in FIG. 2 represent components similar to those illustrated in FIG. 1, with the reference numerals incremented by 100. As shown in FIG. 2A and FIG. 2B, the wireless imaging apparatus 200 can include the removable front imaging module 201, a main module 202 and a locking ring 203. The second portion 212 can be mounted on top of the first portion 211 at an inclined angle, for allowing easier operation of the apparatus 200 by the users. The second portion 212 may comprise a modified mobile device 204 with a touch screen display 205. The orientation of the second portion 212shown in FIG. 2 may be different from the second portion 112 illustrated and described with respect to FIG. 1.

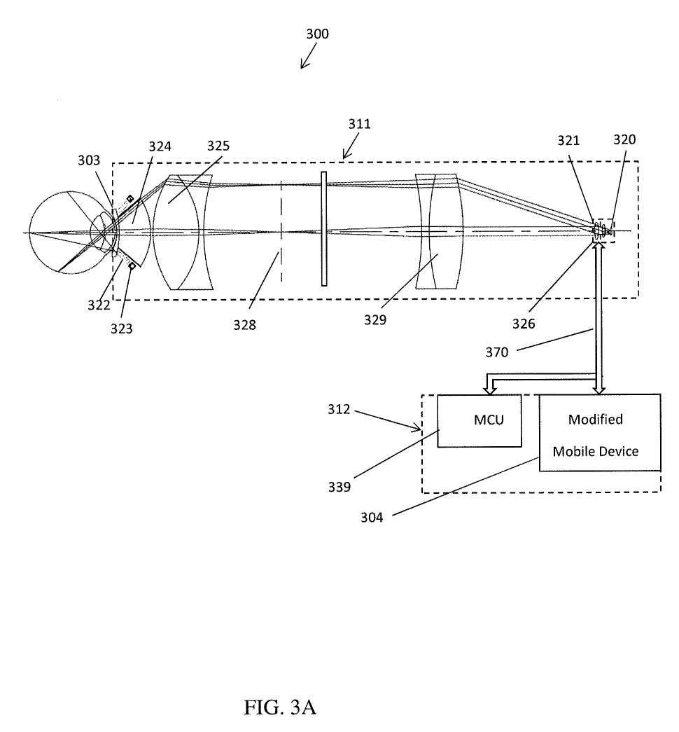

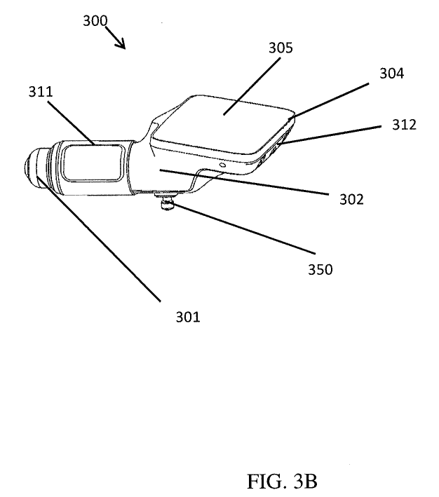

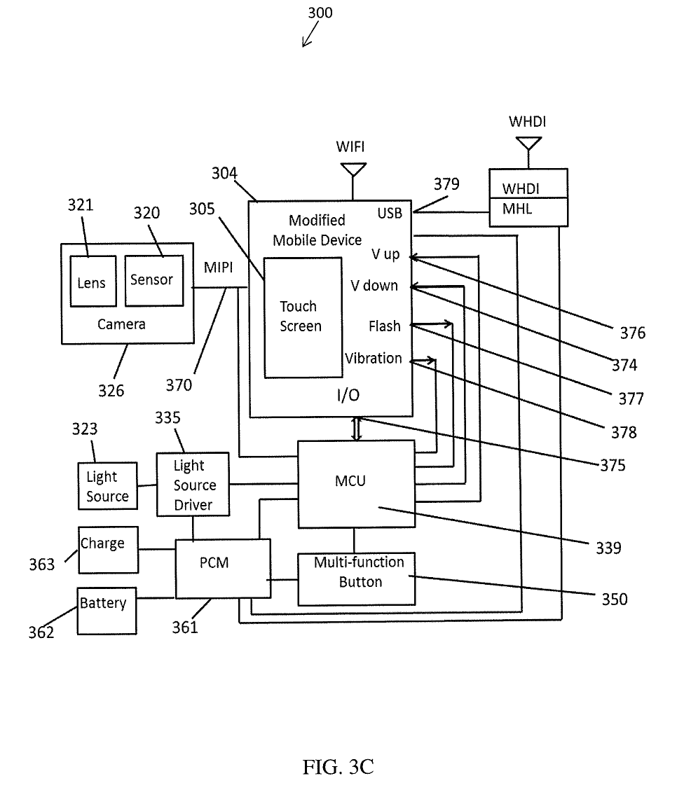

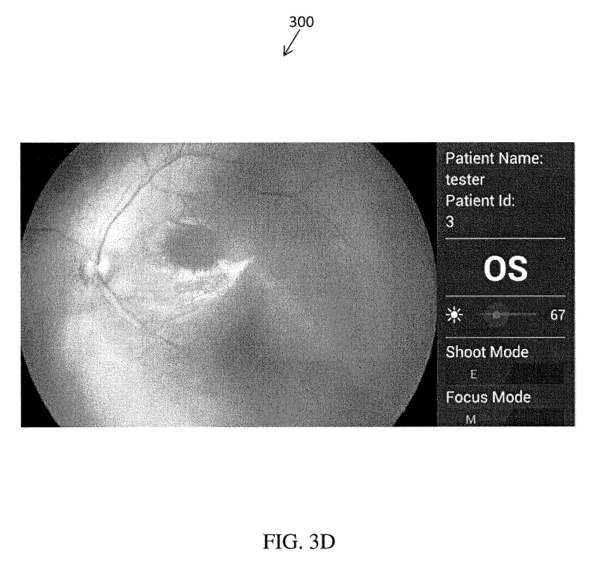

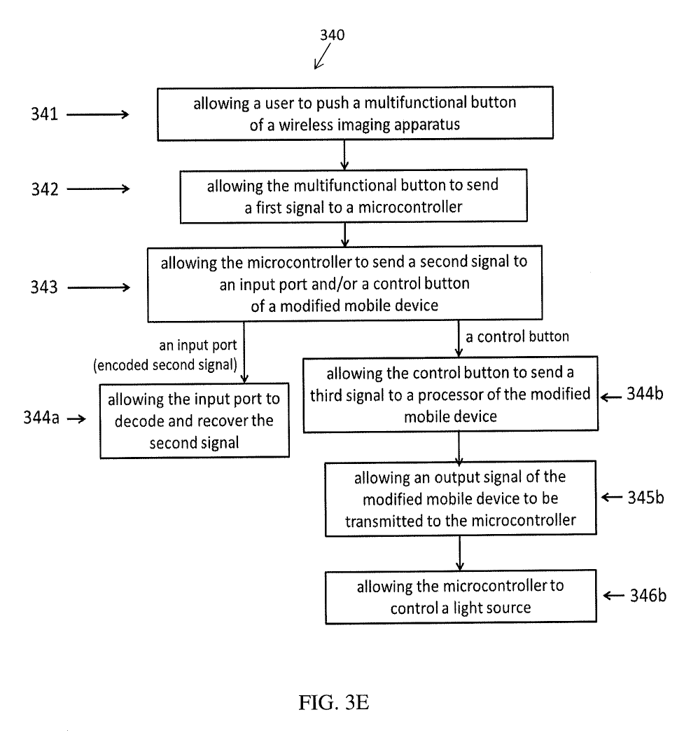

FIG. 3A schematically illustrates an example of an optical system of a wireless imaging apparatus in eye imaging application. Unless otherwise noted, reference numerals used in FIG. 3A-3D represent components similar to those illustrated in FIG. 2, with the reference numerals incremented by 100. The imaging apparatus 300 can comprise a first portion 311 comprising an optical imaging system and an optical illumination system, and a second portion 312 comprising a modified mobile device mobile device 304 and a microcontroller 339. The optical illumination system can comprise a light source 323. In some embodiments, the illumination system can further comprise a light conditioning element, as described in U.S. patent application Ser. No. 14/191,291, titled “Eye Imaging Apparatus with a Wide Field of View and Related Methods”. Illumination light from a light source 323 can be projected from an optical window 303. The light conditioning element 322 may be used to project the light through the designated areas on a cornea and a crystalline lens of an eye, and eventually onto a posterior segment of the eye. An imaging lens 324 behind the optical window 303 may be used to form an image of the posterior segment, which includes a space from a retina to a posterior vitreous chamber of the eye. A first group of relay lenses 325 may be used to relay the image of the posterior segment to a secondary image plane 328. A second group of relay lenses 329 may be added to relay the image from the secondary image plane 328 onto an image sensor 320 of a miniature camera 326. The miniature camera 326 can comprise a lens 321 and an image sensor 320.

The image sensor 320 can be configured to stream real-time video images and/or capture high resolution still images through various pre-programmed functions. The image sensor 320 can include any suitable type of imaging sensor, e.g., a CCD or CMOS sensor. Other types of image sensors may also be used. For example, in some embodiments the image sensor 320 can be a CMOS 8 Mega-pixel image sensor with a format less than 1/1.0, a diagonal dimension less than 10mm. In some other embodiments, the image sensor 320 can be a 13 Mega-pixel CMOS active pixel type stacked image sensor, with a format less than 1/1.0, a diagonal less than 10 mm.

The focusing lens or lenses 321 may be configured to adjust a focal length or a magnification of the imaging apparatus 300. In various embodiments, one or more of the focusing lenses 321 can be configured to be moved or adjusted. For example, one or more of focusing lenses 321 can be translated longitudinally along an optical axis of the optical imaging system with respect to the other focusing lenses 321. Displacing the focusing lenses 321 relative to one another may change the effective optical focal length of the set of focusing lenses 321, which can change the magnification and can result in an optical zoom for the images acquired. Actuators such as voice coils, stepper motors or other types of actuators or combinations thereof may be used to longitudinally translate one or more, or all, of the focusing lenses to change the effective focal length(s) and/or provide zoom. During an imaging procedure, the focusing lens or lenses 321 may be controlled manually or automatically. In the fully automatic mode, the imaging apparatus 300 may automatically look for features in the images and try to adjust the actuator of the focusing lens or lenses 321 to achieve the best focus. In the manual mode, the users may select the area of focus over the live images by using the touch screen monitor 305. The imaging apparatus 300 may adjust the focusing lens or lenses 321 to achieve the best focus in that area and then provide a visual or audible indication when the area is in focus. The image brightness or exposure may also be controlled through automatic or manual mode. In the automatic exposure mode, the users may allow the imaging apparatus to adjust the brightness of the images automatically based on preset imaging criteria. Alternatively, the user may fine tune the exposure by gauging the proper exposure at a selected area in the image, which is often also the area for fine focus adjustment. The overall brightness of the image may be adjusted or set by the users according to their preference. The brightness of the image may be controlled by the sensitivity of the image sensor or luminance of the light source. In some embodiments, the sensitivity of the image sensor can be set to a fixed level when the quality of the images or the noise level of the image is a critical measure. The luminance of the light source can be adjusted to achieve the desired brightness.

For easy control through the modified computing device 304, the miniature camera 326 can be the same miniature camera of the modified mobile device 304 but repositioned outside the modified mobile device 304 and integrated with the optical imaging system of the imaging apparatus 300. Thus, the image sensor 320 can be the same image sensor of the miniaturized camera 326, and the focusing lens or lenses 321 can be the same focusing lens or lenses of the miniaturized camera 326. The miniaturized camera 326 can be connected with the modified mobile device 304 with a cable 370 after being removed outside the modified mobile device 304.

In some other embodiments, the miniature camera 326 can be other miniature cameras that are compatible with the modified computing device 304 and can be configured to be controlled by the central processing unit of the modified mobile device 304 through the cable 370. In some alternative embodiments, the image sensor 320 and at least one focusing lens 321 can be independently selected and configured to be controlled by the modified mobile device 304 through the cable 370. The cable 370 from the miniature camera 326 can split with one branch connected to the modified mobile device 304 and the other branch connected to the microcontroller 339. In some other embodiments, the cable 370 can comprise two cables, one cable connecting the miniature camera 326 to the modified mobile device 304 and the other cable connecting the miniature camera 326 to the microcontroller 339.

When the miniature camera 326 is disposed outside the modified mobile device 304, a conventional cable with a typical length less than 2 mm may not be long enough to connect the miniature camera 326 with the modified mobile device 304. The cable 370 can be a Transmission-Line-Interconnect-Structure (TLIS) configured to have a length between 5 mm and 15 mm. In some embodiments, the cable 370 can be configured to connect the miniature camera 326 to the modified mobile device 304. In some other embodiments, the cable 370 can be configured to connect the miniature camera 326 to both the modified mobile device 304 and the microcontroller 339. In some alternative embodiments, the cable 370 can be configured to connect the miniature camera 326 to the microcontroller 339.

The cable 370 can be configured to meet the interface requirements under Mobile Industry Processor Interface (MIPI) specifications which support a full range of application requirements in mobile devices. In various embodiments, the cable 370 can be configured to meet MIPI specifications supporting camera and display interconnections including but not limited to MIPI's Camera Serial Interface-2 (CSI-2), and MIPI's Camera Serial Interface-3 (CSI-3) in order to meet the demanding requirements of low power, low noise generation, and high noise immunity. For example, the cable 370 can be configured to have a reference characteristic impedance level that is about 100 Ohm differential, about 50 Ohm single-ended per Line, and about 25 Ohm common-mode for both Lines together according to MIPI specifications. The reference characteristic impedance can be affected by the cable parameters such as line width, distance between lines, copper thickness, substrate thickness, etc. The parameters of the cable 370 can be determined by using TLIS simulation software, for example, Polar Si8000 by Polar Instruments. In some embodiments, for example, the cable 370 can have a substrate thickness between 0.05 mm to 0.2 mm, and a copper thickness between 5 um to 50 um. In some other embodiments, the cable 370 can have parameters with other values that meet MIPI specifications.

In some other embodiments, the light source 323 can be the flash light of the modified mobile device 304 but repositioned outside the modified mobile device 304 and integrated with the optical illumination system of the imaging apparatus 300. The light source 323 can be connected with the modified mobile device 304 with another cable. In some alternative embodiments, the light source 323 can be other light sources that can be configured to be controlled by the central processing unit of the modified mobile device 304. In some other embodiments, the light source 323 can be configured to be controlled by the microcontroller 339. In some more embodiments, the light source 323 can be configured to be driven by an independent driver 335, and the microcontroller 339 can be configured to control the driver 335.

FIG. 3B is a perspective view that schematically illustrates a wireless imaging apparatus 300 with a multi-functional control button 350. The imaging apparatus 300 may further comprise a multi-functional button 350 disposed on the housing of the apparatus 300. The multi-functional button 350 can be configured to control the light source 323, the actuator of the focusing lens or lenses 321, and the image sensor 320. In some embodiments, for example, the multi-functional button 350 can be disposed on the cylindrical portion 311 of the housing of the imaging apparatus 300, thus allowing easy operation of the user with only one hand. For example, as shown in FIG. 3B, the imaging apparatus 300 may be held by the user using four fingers, while leaving the index finger free to operate the multi-functional button 350. The multi-functional button 350 can enable the operation of the imaging apparatus 300 with only one hand. The multi-functional button 350 can comprise electrical switches to control the light source 323, the actuator of the focusing lens or lenses 321, and the image sensor 320. Therefore, the multi-functional button 350 can allow the user to control the focus, the light intensity, and the image capturing process by using just one finger. For example, in some embodiments, the intensity level of the light source 323 may be adjusted by pushing the multi-functional button 350 to the left and/or right, and the actuator of the focusing lens or lenses 321 may be adjusted by pushing the multi-functional control button 350 up and/or down. In other embodiments, the intensity level of the light source 323 may be adjusted by pushing the multi-functional button 350 up and/or down, and the actuator of the focusing lens or lenses 321 may be adjusted by pushing the multi-functional control button 350 left and/or right. In some embodiments, the multi-functional button 350 may also be used as a trigger for the image sensor 320 by pushing the multi-functional button inwardly. Other variations of using the multi-functional button 350 to control the imaging apparatus 300 may also be suitable.

FIG. 3C schematically illustrates a block diagram of an example of an electronic system of the wireless imaging apparatus 300 in an eye imaging application. In various embodiments, the imaging apparatus 300 can comprise a modified mobile device 304 with a built-in data communication capability. The modified mobile device 304 may be based on a modification of a conventional mobile device comprising a low power central processing unit (CPU), a graphic processing unit (GPU), an operating system (such as Android or iOS mobile operating systems), a touch screen display, a miniature camera, Input/output ports, as well as other modules for wireless connectivity. The imaging apparatus 300 can utilize the built-in high speed data communication capability and high computing power of the modified mobile device 304. Because the conventional mobile device may be primarily configured to communicate audio signals, the conventional mobile device may only have limited input/output communication ports. For example, a smart phone may only have a few in/out communication ports such as an input port for charging power, an input/output port for a microphone/speaker phone, and a few control buttons such as volume adjustment buttons. On the other hand, an imaging capturing process can be complicated, including precise control and synchronization of the light source, the focusing lens, and the image sensor. Thus, the conventional mobile device may not be capable of controlling the image capturing process involving multiple devices disposed outside the mobile device without modification. For example, the smart phone will not be capable of controlling and synchronizing the light source 323, the focusing lens 321, the image sensor 320 and the multi-functional button 350. Therefore, the conventional mobile device may have to be modified in order to control the image capturing process, including control and synchronization of the light source 323, the miniature camera 326, and the multi-functional button 350.

As shown in FIG. 3C, the conventional mobile device can be modified to control the image capturing process. The modification of the conventional mobile device can include the modification of a hardware structure. For example, the miniature camera 326 can be removed to be outside the modified mobile dive 304 and the cable 370 can be added as discussed above. An input/output port 375 of the modified mobile device 304 can be modified to be connected to a device, for example, the microcontroller 339, which is disposed outside the modified mobile device 304.

The modification of the conventional mobile device can further include modification of a non-transitory, computer-readable storage medium storing a set of instructions in the modified mobile device 304. The instructions, when executed by a processor of the modified mobile device 304, can be modified to cause the processor to control the image capturing process. In some embodiments, the input/output port 375 of the conventional mobile device can be modified to control the imaging capturing process by modification of the instructions in the non-transitory, computer-readable storage medium of the modified mobile device 304, in addition to connecting the input/output port 375 to the microcontroller 339. In some other embodiments, a control button (e.g., a volume up button 376 or a volume down button 374), and/or an output signal (e.g., a flash signal 377 or a vibration signal 378) of the modified mobile device 304 can be modified to control the image capturing process by modification of the instructions related to the control button and/or the output signal, in addition to modification of the connection of the control button and/or the output signal. Overall, in order to control the image capturing process including control and synchronization of the light source 323 and the miniature camera 326, the modified mobile device 304 can include, but not be limited to, the modification of a structure of the mobile device 304, the modification of instructions stored in non-transitory, computer-readable storage medium of the mobile device 304, and any combination thereof.

The imaging apparatus 300 can comprise a modified mobile device 304, configured to control and synchronize the miniature camera 326 and the light source 323. The imaging apparatus 300 can be configured to receive the images from the image sensor 320 in real time. The live images can be displayed on the touch screen monitor 305 of the modified mobile device 304. In some embodiments, the image sensor 320 and the image capturing features can be controlled through the input/out port 375 of the modified mobile device 304. In some other embodiments, the image sensor 320 and the image capturing features can by controlled by the control buttons (e.g., a volume up button 376 or a volume down button 374) of the modified mobile device 304. In some alternative embodiments, the image sensor 320 and the image capturing features can be controlled on the touch screen monitor 305, and/or by voice command functions of the modified mobile device 304. The imaging apparatus 300 can also be configured to exchange data and communicate with other electronic devices through wired or wireless communication systems, such as WiFi or 3G standard telecommunication protocols.

In various embodiments, the imaging apparatus 300 can comprise a microcontroller (MCU) 339 connected to the modified mobile device 304 (e.g., the modified smart phone) to further expand the control capability and flexibility of the modified mobile device 304. The MCU 339 can communicate with the modified mobile device 304, the miniature camera 326, the light source 323, and the multi-functional button 350. The MCU 339 may comprise a central processing unit, a memory and a plurality of communication input/output ports. The central processing unit may range from 16-bit to 64-bit in some embodiments. The MCU 339 may further comprise any suitable type of memory device, such as ROM, EPROM, EEPROM, flash memory, etc. The MCU 339 may comprise analog-to-digital converters and/or digital-to-analog converters in various embodiments. The MCU 339 may comprise input/output ports such as I2C, Serial SCCB, MIPI and RS-232. In some embodiments, USB or Ethernet ports may also be used.

In some embodiments, the MCU 339 may be connected to the light source 323, the image sensor 320, and the actuator of the focusing lens or lenses 321 through the plurality of communication input/output ports. In some other embodiments, the imaging apparatus 300 may further comprise an independent driver 335 to drive the light source 323 when the required electrical power of the light source 323 is substantially higher than the power of a conventional light source of a mobile device. The driver 335 may comprises an integrated multi-channel current-source type driver chip in some embodiments. The driver chip may modulate the light output or the brightness of the light source based on configurations of pulse-width-modulation. As a result, the independent driver 335 can be configured to drive a more powerful light source than the conventional light source in typical mobile devices. In addition, the driver 335 can be configured to drive multiple light sources 323 at the same time. The driver 335 may be powered by a battery in the modified mobile device 304 or by a separate battery with larger capacity and larger current. The control of the light source 323, as well as the control of the driver 335, may be carried out through the MCU 339. In some embodiments, the MCU 339 can be connected with the multi-functional button 350, which is configured to control the light source 323, the actuator of the focusing lens or lenses 321 and/or the image sensor 320. After the user pushes the multi-functional button 350, the MCU can be configured to receive a trigger signal in response to the pushing action, and send a second electrical signal to the modified mobile device 304 in response to the trigger signal.

The MCU 339 and the modified mobile device 304 can be configured to communicate to each other in order to control and synchronize the operation of the light source 323 and the image sensor 320. The MCU 339 and the modified mobile device 304 can be further configured to control the actuator of the focusing lens or lenses 321 in front of the image sensor 320 to adjust the effective focal length and/or the magnification of the imaging apparatus 300.

Referring to FIG. 3C, the MCU 339 can be configured to communicate with the modified mobile device 304 through the input/output port 375. Communication between the MCU 339 and the modified mobile device 304 may be realized through the input/output port 375 of the modified mobile device 304 (e.g., a modified smart phone). The input/output port 375 of the modified mobile device 304 can be modified to control the imaging capturing process by modification of instructions stored in the non-transitory medium of the modified mobile device 304 in order to convert a command signal to a data format recognizable by the input/output port 375. For example, a microphone/speaker port 375 of the modified mobile device 304 may be used to provide such communication. The microphone/speaker port 375 may be primarily configured to communicate an audio signal. Therefore, the microphone/speaker port 375 may have to be modified in order for the MCU 339 and the modified mobile device 304 to communicate a command signal other than the audio signal. The microphone/speaker port 375 can be modified to transmit the command signal by encoding the command signal into the audio signal, and recovering the command signal by decoding the audio signal. The encoding of the command signal and decoding the audio signal may employ a variety of conversion algorithms.

When a user pushes in a trigger button on the multi-functional button 350, the multi-functional button 350 can send a trigger signal in response to the pushing action. For example, the trigger signal can be a five digit command signal. The five-digit command signal may be read into the MCU 339. In order to transfer the five-digit command signal, the MCU 339 may comprise instructions to encode the five-digit command signal in the frequency of an audio signal. In some embodiments, a character encoding scheme American Standard Code for Information Interchange (ASCII) can be used. The ASCII can represent each digit of the five-digit signal by 7-bit binary integers. The 7-bit binary integers can be encoded in the frequency of audio signals. Then, the MCU 339 may send a series of electric pulses representing the five-digit signal, encoded in the frequency of audio signals, to the microphone/speaker port 375 of the modified mobile device 304. The modified mobile device 304 (e.g., modified smart phone) can receive the audio signals as if the audio signals are voice calls. The microphone/speaker port 375 of the modified mobile device 304 can be modified to include instructions to decode the received audio signals, thereby recovering the five-digit command signal. The encoding and decoding of the audio signals may employ many algorithms, including, but not limited to, Fourier Transform, Fast Fourier Transform (FFT), complex modified discrete cosine transform (CMDCT), Pulse-Width Modulation (PWM), etc.

In some embodiments, FFT can be used in the signal processing. FFT is an algorithm to compute the discrete Fourier transform (DFT) and its inverse. The DFT is obtained by decomposing a signal into components of different frequencies. An FFT can be used to compute the same result more quickly. FFT can be realized by computing the DFT at many discrete points, such as 26, 32, 64, 128 points, etc. For example, a signal with a digit “A” from the multi-function button 350 can be sent to the MCU 339, and “A” can be represented by ASCII as “1000001B”. The digit “A” can be encoded in the frequency of an audio signal. For example, the MCU 339 may send a series of electric pulses representing the signal “A”, encoded in the frequency of audio signals such as A(t)=1*sin(7X)+0*sin(6X)+0*sin(5X)+0*sin(4X)+0*sin(3X)+0*sin(2X)+1*sin(X), where “X” represents a fundamental frequency of the audio signals. Typically, the microphone/speaker port 375 can respond to audio signals in the frequency range between 20 Hz to 20K Hz. Audio signals can be sampled at 44.1 kHz, 48 kHz, 88.2 kHz, 96 kHz, 192 kHz, etc. For example, when 32 points FFT algorithm is used, the fundamental frequency “X” can be calculated as “44.1 kHz/32=1.378 kHz”. After receiving the audio signals, the microphone/speaker port 375 can be modified to decode the received audio signals. For example, the microphone/speaker port 375 can be configured to perform FFT algorithm to the audio signal “A(t)”, and thereby recovering the command signal as “A”=1000001B.

In the other direction, a command from the modified mobile device 304 (e.g., a command from the touch screen 305), can be encoded as audio signals and sent out to the microphone/speaker port 375. The microphone/speaker port 375 can send the encoded audio signals to the MCU 339. The MCU 339 can receive the audio signals and recover the command signal, for example, by FFT decoding. The recovered command can be used by the MCU 339 to control the light source 323, or the actuator of the focusing lens or lenses 321, or the image sensor 320.

Though the microphone/speaker port 375 of the modified mobile device 304 may be used in the communication to the MCU 339 in some embodiments, other standard input/output ports of the modified mobile device 304 may be used as well. The MCU 339 may convert the command signal into various formats of signals recognizable by other input/output ports. The other input/output ports may be modified to recover the command signal by applying various conversion algorithms.

As shown in FIG. 3C, communication between the MCU 339 and the modified mobile device 304 may be realized through a control button (e.g., the volume up control button 376, or the volume down button 374), or an output signal (e.g., the vibration signal 377, or the flash signal 378) of the modified mobile device 304 (e.g., a modified smart phone). The control button 376/374 and the output signal 377/378 can be modified to be connected with the MCU 339 and configured to control the image capturing process.

In some embodiments, the control button, such as the volume up 376 or the volume down button 374, can be modified to be connected to the MCU 339. The volume up control button 376 or a volume down button 374 can be operational through a mechanical relay. The mechanical relay may comprise a mechanical structure that translates a motion of the user into a motion that one of the electrical switches on the modified device 304 is configured to respond to. When a user pushes in the multi-functional button 350, the multi-functional button 350 can be configured to send the MCU 339 a trigger signal, which is a first signal, in response to the pushing action. The MCU 339 can send a second signal to the control button 376 or 374 of the modified device 304, in response to the first signal. The control button 376 or 374 can comprise an electrical switch that is configured to respond to the second signal from the MCU 339, thereby sending a third signal to the processor of the modified mobile device 304. The control button 376 or 374 can inform the modified mobile device 304 to control the miniature camera 326 by starting the instructions stored in the non-transitory medium for image capturing. The transmission of the trigger signal through the control button 376/374 can be much faster than through the input/output port 375. During the image capturing process, both the object and the imaging apparatus may move slightly, which could result in misalignment and reduce image quality. After the user pushes in the trigger button, the faster the trigger signal is transmitted, the less chance that misalignment from the object or the apparatus movement might occur. Therefore, the modification of the control button 376/374 to control the imaging process can reduce misalignment and increase image quality.

Moreover, an output signal of the modified mobile device 304 such as a flash signal 377 or a vibration signal 378 can be modified to connect to the MCU 339. In the image capturing process, the activation of the light source 323 may have to be synchronized with the shutter of the image sensor 320. The synchronization can be carried out through the communication of the MCU 339 with the modified mobile device 304 by modifying an input signal of the modified mobile device 304. In some embodiments, the sequential illumination method can be employed while multiple light sources are activated time-sequentially in order to obtain high quality images. The precise synchronization of the light source 323 with the shutter of the image sensor 320 can be particularly important under sequential illumination. In addition to modifying the control button 376/374 of the modified mobile device 304, the output signal (e.g., the vibration signal 377 or the flash signal 378) of the modified mobile device 304 can be modified to achieve the precise synchronization.

The imaging apparatus 300 can employ sequential illumination to overcome scattering problems and obtain high quality images. In some embodiments, the imaging apparatus 300 can comprise the light source 323, which can further include a plurality of light emitting elements configured to illuminate different portions of an object time-sequentially. The image sensor 320 can be configured to receive a plurality of images with a same wide field of view through the optical imaging system while each portion of the object is illuminated time-sequentially. The plurality of images may be processed to create a single clear image.

In various embodiments, different portions of the object can be selectively illuminated more than other portions. The portion selected for increased illumination can be changed so as to provide increased illumination of the different portions at different times. Such selective illumination by selectively activating the light emitting elements 323 can be synchronized with the image sensor 320 to obtain the images captured at those times. Accordingly, images can be obtained at these different times and used to produce a composite image that has less haze and glare. In some embodiments, a driver 355 can be used to activate the light emitting elements 323 to direct light from a selected emitter or emitters and not from the other or otherwise can selectively modulate the emitters. In some embodiments, simply more light from the selected emitter or emitters is provided in comparison to the other emitter. In various embodiments, shutters, light valves, and/or spatial light modulators can be employed to control the amount of light from each of the light emitting elements 323. Although one emitter at a time was described above as being activated, more than one light emitter can be activated at a time. In various embodiments, more light is provided by a subset of the total number of emitters 323 so as to illuminate a portion of the object more than one or more other portions. An image can be recorded. Subsequently, a different subset of the total number of emitters can be selected to illuminate another portion of the object or illuminate that portion more than others. Another image can be recorded. This process can be repeated multiple times in various embodiments. For example, 2, 3, 4 or more subsets may be selected at different times or for providing the primary illumination. Images of the object may be obtained at the different times. These images or at least portions of these images may be employed to form a composite image of the object.

Because the object or the imaging apparatus may be moved slightly during the imaging process, the features from the several partial images may not overlap precisely. The extended area from the border of each quarter may be used to allow the proper adjustment and re-alignment of the images. In some embodiments, in order to align the images taken time sequentially, one or more additional images may be captured with all of the light emitting elements activated at the same time, in addition to the multiple images taken time-sequentially as described above. This image can be obtained using the same optical imaging system having the same field of view as was used to obtain the plurality of images obtained with time-sequential illumination. Although such image may be hazy or with glare, it may contain the unique graphic reference features of the whole imaging area or the entire field of view. Using this image as a reference image to coordinate, each of the rest of the partial images may be aligned with the reference image. The clear composite image could then be formed from the multiple images after proper adjustment of the locations.

Although in the example embodiment described above, a single reference image was obtained with all the light emitters activated to assist in alignment of the other images, in other embodiments less than all light emitters may be illuminated. Accordingly, one or more reference images can be employed to align images of sections obtained using time-sequential illumination. To generate a reference image, the multiple sections are illuminated and an image is capture by the optical imaging system and sensor. This reference image will depict the sections and their positional relationship, and will contain reference features that can be used to align separate images of the separate sections. Although reference images can be obtained by illuminating all of the sections, not all the sections need to be illuminated at the same time to produce reference images that can assist in alignment. These reference images can be captured using the same optical imaging system having the same field of view as was used to obtain the plurality of images captured during time-sequential illumination. However, in alternative embodiments, reference images can be captured by other optical imaging systems and sensors. Additionally, reference images can be captured with different fields-of-view. Other variations are possible.

Accordingly, a sequential illumination method can be employed to obtain high quality images with a wide field of view. The method comprises activating a plurality of light emitting elements 323 time-sequentially to illuminate different portions of an object, imaging the object through an optical imaging system, and receiving a plurality of images of the object through the optical imaging system and sensor while different portions of the object are illuminated time-sequentially. The images are captured by the image sensor 320 and processed to create a single clear image. The sequential illumination method may be applied when different numbers of the light emitting elements are used. The possible examples include 2 elements, 3 elements, 4 elements, 6 elements, 8 elements or even more elements. The light emitting elements need not be individually activated. In some embodiments, pairs may be activated at a time. Similarly, 3, 4, or more may be activated at a time. Other variations are possible. In various embodiments, the rate of frequency of the time-sequential capturing is determined by the image capturing rate. In some embodiments, the imaging apparatus 300 can be configured to capture each image between 50 ms to 150 ms, or 200 ms, or 300 ms, or 600 ms.

The image capturing process employing sequential illumination can be a complicated process. A plurality of images can be captured by the image sensor 320 time-sequentially to create the single clear image. It is advantageous to complete the imaging capturing process in a short time period. Otherwise, both the object and the imaging apparatus 300 may move, which may result in misalignment, even focus shift, thus severely degrading the image quality. In some embodiments, a burst mode built in the modified mobile device 304 can be used in sequential illumination. Under burst mode, the modified mobile device 304 can be configured to capture several images continuously in a very short time period.

Burst mode can be utilized under sequential illumination to ensure the image quality. As discussed above, when the user pushes in the multi-functional button 350, MCU 339 can send a trigger signal to the control button 376 or 374. The control button 376 or 374 can send a second signal to the processor of the modified mobile device 304 to control the miniature camera 326 by starting the instructions to capture the image. In other words, the input signal from the control button 376 or 374 is modified to be used to start the image capturing process in order to synchronize the image sensor 320 and the light source 323. In some embodiments, a reference image can be captured first. Then a first image can be captured after activating a first light emitting element, and a second image can be captured after turning off the first light emitting element and activating a second light emitting element, and so on.

However, the time duration for capturing the first reference image may vary in a large range under burst mode. Because of the illumination condition varies, the miniature camera may need to be calibrated before taking the reference image, and the time of the calibration process may vary as well. For example, the reference image may be captured by the image sensor 320 anywhere between 100 ms to 600 ms after the second signal sent from the control button 376 or 374. The uncertainty in the time duration before the reference image being captured can cause inaccuracy in synchronization, thus degrading of the image quality.

Under the sequential illumination in burst mode, more precise control may be needed by modifying at least one output signal such as the flash signal 377, the vibration signal 378 or other output signals. Though the large uncertainty of time duration for capturing the reference image exists, after the first reference image, the time duration between each subsequent image capturing can be about the same, for example, about 15 ms, 30 ms, 50 ms, 125 ms, or 150 ms, or any values therebetween. Thus, if an output signal (e.g., the flash signal 377, the vibration signal 378) of the modified mobile device can be generated after the first reference image is captured and sent to the MCU 339, the activation of each light emitting element of the light source 323 can be precisely synchronized with the shutter of the image sensor 320 in each image capturing process to provide the required light intensity at the precise time.

In some embodiments, a flash signal 377 of the modified mobile device 304 can be modified to reduce the uncertainty and increase the accuracy of the synchronization. In general, the time duration between the ending of an image capture and the beginning of the next image capture is about the same after the reference image was captured as the illumination condition was calibrated. For example, the reference image may be captured by the image sensor 320 anywhere between 100 ms to 600 ms after the signal sent by the volume up/down button 376/374, and the time between each image capture may be about 125 ms after the reference image is captured. In some embodiments, for example, after the reference image was captured by the image sensor 320, an electrical switch to generate a flash signal 377 can be triggered. The instructions stored in the non-transitory medium for image capturing can be modified to receive the flash signal 377 and cause the processor to activate the first light emitting element in about 125 ms. In about another 125 ms, the instructions can cause the processor to turn off the first light emitting element and activate the second light emitting element. The process can continue to go on. In this way, the activation of each light emitting element can be accurately synchronized with the shutter of the image sensor 320, thus obtaining high quality images.

It is appreciated that the number of light emitting elements may vary, the duration before the reference image capture may vary, and the duration between each image capture after the reference image capturing may vary as well in some other embodiments.

In some alternative embodiments, a vibration signal 378 of the modified mobile device 304 can be used instead of the flash signal 377 to increase the accuracy of the synchronization. For example, after the reference image was captured by the image sensor 320, an electrical switch may be modified to generate a vibration signal 378. The physical vibration structure, for example, a motor, can be removed to avoid physical vibration which could result in misalignment. However, an electrical vibration signal 378 can be generated in response to the signal sent from the volume up/down button 376/374. The instructions stored in the non-transitory medium for image capturing can be modified to receive the vibration signal 378 and cause the processor to activate the first light emitting element in about 125 ms. The instructions can cause the processor to turn off the first light emitting element and active the second light emitting element in about another 125 ms. The process can continue until all of the light emitting elements are activated. Therefore, the activation of each light emitting element can be accurately synchronized with the shutter of the image sensor 320 by modifying the output signal, such as the flash signal 377, the vibration signal 378, under burst mode in sequential illumination.

The output signal (e.g., the flash signal 377, the vibration signal 378, etc.) can be modified as a handshake signal to increase the efficiency and speed of communication in some other embodiments. For example, the MUC 339 can communicate with the modified mobile device 304 through an audio port 375. The audio port 375 is a serial port that can use a handshake signal in the interface to pause and resume the transmission of data. For example, before the MCU 339 starts to transmit signals to the modified mobile device 304 through the audio port 375, the MCU 399 can send a signal, for example, a Request to Send (RTS) signal to one of the control buttons (e.g., the volume up/down button 376/374) of the modified mobile device 304. In response to the RTS signal, the volume up/down button 376/374 can be modified to send a second signal to start instructions to receive and decode the transmission from the MCU 339. Then a circuit to generate a vibration signal 378 can be modified to generate an electrical vibration signal 378 in response to the signal sent from the volume up/down button 376/374. The modified mobile device 304 can be modified to send the electrical vibration signal 378 as Clear to Send (CTS) signal to MCU 339. After the MCU 339 receives the CTS signal, the MCU 339 can immediately start transmission of data through the audio port 375. Without RTS/CTS signals, the modified mobile device 304 may require a lot of time and resources to constantly monitor the audio port 375 to determine whether MCU 339 will transmit data. By modifying and using the output signal (e.g., the flash signal 377, the vibration signal 378, etc.) as the RTS/CTS signal, the communication efficiency, speed and reliability between the audio port 375 and the MCU 339 can be increased.

The wireless imaging apparatus 300 can comprise an electronic system which is built around the modified mobile device 304. The live images captured by the imaging sensor 320 can be transmitted to the modified mobile device 304, e.g., in the form of RAW data format. The live images can be processed and calibrated to form a standard video stream, which may be displayed on the touch screen monitor 305 of the modified mobile device 304. The same video stream may be transmitted out of the device 304 in real time through the USB port 379. The USB port 379 can be connected by Mobile High-Definition Link (MHL), which is an industry standard interface to connect the modified mobile device 304 to high-definition displays. In some embodiments, Wireless Home Digital Interface (WHDI) specification can be used to transmit an uncompressed high-definition digital video wirelessly to any compatible display devices in hospitals or medical facilities.

In some embodiments, the wireless imaging apparatus 300 can further comprise a power management module 361. The power management module 361 can be charged by a charge 363 or a battery 362. The power management module 361 can provide power to the electronic system of the wireless imaging apparatus 300 including the modified mobile device 304, the MCU 339, the multifunctional button 350, the light source driver 335, and the MHL connected with the USB port 379, etc.

FIG. 3D is a screen shot of a user interface of a wireless imaging apparatus 300 in eye imaging application according to some embodiments. The wireless imaging apparatus 300 can comprise a user interface to allow the user to preview the image and control the image capturing process. The user interface may allow the user to input the patient name and patient identification number. The imaging apparatus 300 can be configured to communicate with a base station and a computing device in a hospital or medical clinic wirelessly. The information of the patient name and patient identification number can be transmitted to the imaging apparatus wirelessly as well. After the user places the imaging apparatus on an object, the user interface can allow the user to preview the image. The user may be required to take several images of the object in several different fields of view according to directions from a physician. The user may perform precise alignment, adjust the focus and light intensity during the preview process. The user interface may allow the user to select image capturing mode, for example, sequential illumination under burst mode. The user interface may also allow the user to view the focusing status. When the user finishes the alignment and adjustment, the user may push the multi-functional button and capture the image.

FIG. 3E is a flow diagram that schematically illustrates an example of a method 340 of control of an imaging capturing process of a wireless imaging apparatus comprising a light source, a miniature camera, and a modified mobile device according to various embodiments.

The miniature camera can be disposed outside the modified mobile device and connected to the modified mobile device by a cable. The wireless imaging apparatus can comprise a microcontroller in some embodiments. The wireless imaging apparatus can further comprise a multi-functional button in some embodiments.

The method can comprise allowing a user to push the multifunctional button of the wireless imaging apparatus, as shown in block 341. The user may push the multi-functional button in to trigger the image capturing process in some embodiments. The user may push the multi-functional button up and down, or left and right to adjust focus and light intensity in some other embodiments.

The method can comprise allowing the multifunctional button to send a first signal to the microcontroller in response to the pushing action, as shown in block 342. The method can further comprise allowing the microcontroller to send a second signal to an input port and/or a control button of the modified mobile device in response to the first signal, as shown in block 343.

In some embodiments, the method can comprise allowing the microcontroller to send the second signal to the modified mobile device through an input port of the modified mobile device. For example, the method may comprise allowing the microcontroller to encode the second signal in an audio signal, and transmit the audio signal to the microphone port of the modified mobile device. The method may further comprise allowing the microphone port of the modified mobile device to decode the audio signal and recover the second signal, as shown in block 344a.

In some other embodiments, the method can comprise allowing the microcontroller to send the second signal to the modified mobile device through a control button of the modified mobile device. The method may further comprise allowing the control button to send a third signal to the processor of the modified mobile device to control the miniature camera by starting the instructions for image capturing, in response to the second signal, as shown in block 344b.

In some embodiments, the method may further comprise allowing an output signal of the modified mobile device to be generated and transmitted to the microcontroller, as shown in block 345b. For example, the method may comprise allowing an output signal to be generated after the instructions for imaging capturing started and the reference image captured, and allowing the output signal to be transmitted to the microcontroller. The method may further comprise allowing the microcontroller to send another signal to the light source to activate the light source, in response to the output signal from the modified mobile device, as shown in block 346b.

In some other embodiments, the method may comprise allowing the microcontroller to send a first handshake signal to a control button, for example, a Request to Send (RTS) signal. The method may further comprise allowing an output signal to be generated and sent back to the microcontroller as a second handshake signal, in response to the first handshake signal. For example, the control button can be modified to send another signal to start instructions to receive and decode the transmission from the microcontroller. Then a vibration signal can be generated and sent to the microcontroller as Clear to Send (CTS) signal. The method may allow the microcontroller to start transmission of data after receiving the CTS signal.

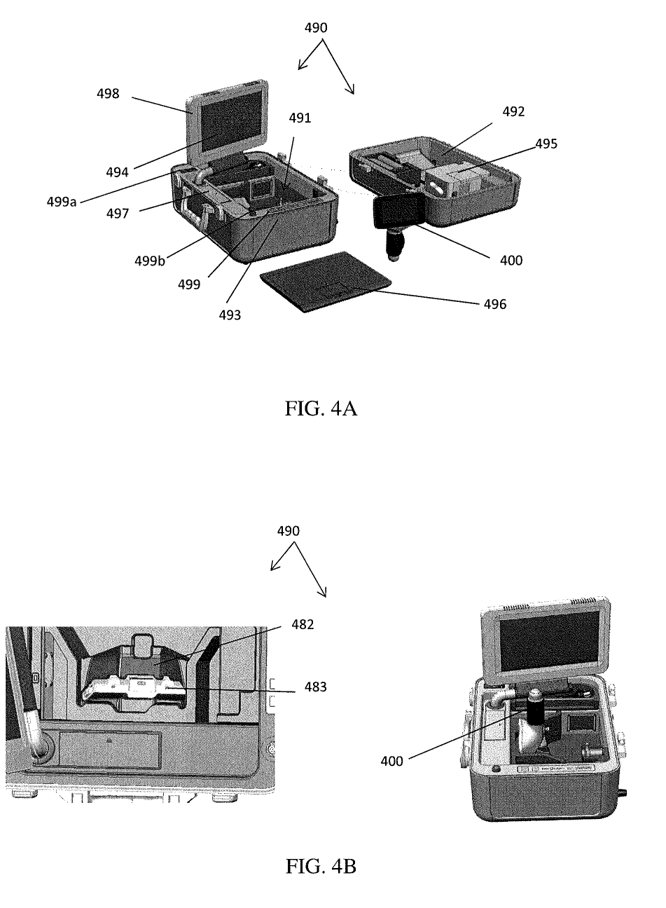

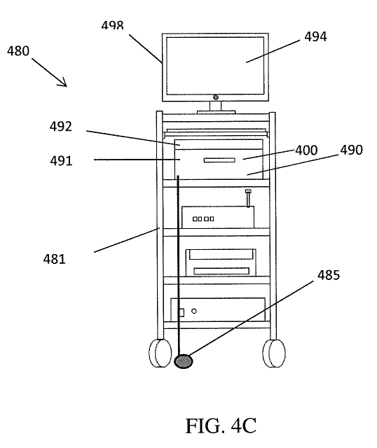

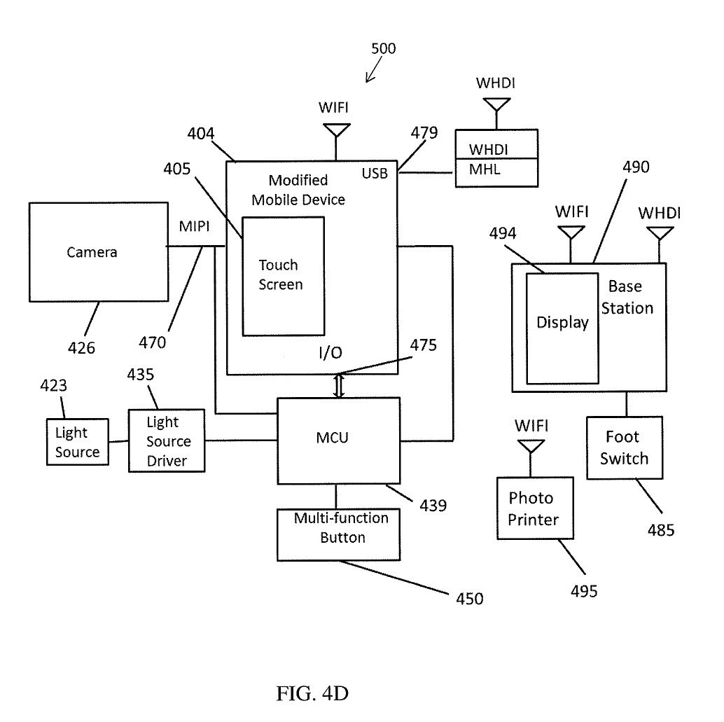

FIG. 4A is a perspective view that schematically illustrates a base station for the imaging apparatus 400 according to various embodiments. The base station 490 can comprise an electronic system including a control panel 499, a computing module 498, a display monitor 494, a communication module 493, and a printer 495. The control module 499 can include a power entry module 499a, a power on/off switch 499b, and a plurality of wires. The communication module 493 can be disposed underneath the control panel 499 and configured to transmit to and receive signals from the imaging apparatus 400. To power up the base station 490 by AC source, a power cord can be first plugged into the power entry module 499a in one end, and plugged into the AC power outlet in another end. By pushing down the power on/off switch 499b, the whole electronic control panel 499 in the base station/carrying case 490 can be powered up. The computing module 498, the display monitor 494, and printer 495 can be configured to receive images from the imaging apparatus 400 wirelessly. In various embodiments, the base station 490 can be configured to receive data input via, for example, the wireless keyboard 496 as well as images from the imaging apparatus 400. The display monitor 494 can be used to display and review the patients' images. The printer 495 can be used to print the report and the images.

In some embodiments, the base station can be the carrying case 490. The base station/carrying case 490 can have a main portion 491 having an open inner region for storage, and a cover 492. In some embodiments, the base station/carrying case 490 can have at least one of a computing module 498, a display monitor 494, a printer 495, or a charging station (not shown) integrated to the carrying case 400. The display monitor 494 and printer 495 can be configured to receive images from the imaging apparatus 400. The charging station can be configured to charge the imaging apparatus 400. The carrying case 400 can be configured to house the imaging apparatus 400 as well as the display monitor 494, a wireless keyboard 496, a removable electronic data storage unit 497, etc. In some embodiments, the display monitor 494 can be integrated with the computing module 498 as one unit. In some embodiments, the display monitor 494 can have a touch screen function. In some embodiments, the removable electronic data storage unit 497 can be a custom-built hard disk drive, which can be removable such that the removable electronic data storage unit 497 can be taken out to be placed in a secure location for data safety.

The imaging apparatus 400 can be configured to communicate with the base station/carrying case 490. The imaging apparatus 400 may be carried in the carrying case 490 because the imaging apparatus 400 is relatively compact and easy to carry. For example, in some embodiments, the carrying case 490 can have dimensions less than about 600 mm×400 mm×300 mm and can weigh less than about 20 kg. In some embodiments, for example, the carrying case 490 (with or without the imaging apparatus 400 inside) can be between (600 mm and 300 mm)×(400 mm and 200 mm)×(300 and 150 mm). Similarly, in some embodiments the carrying case 490 can have a volume less than 72,000 cm3. In some embodiments the carrying case 490 can have a volume between 72,000 cm3 and 9000 cm3. Also, the carrying case 490 can weigh between about 10 kg and about 20 kg in some embodiments, or between about 5 kg and about 20 kg, in some embodiments. Sizes outside these ranges for the carrying case 490 are also possible.

Referring to FIG. 4A, after computing module 498, the printer 495 and the imaging apparatus 400 are powered up, the computing module 498 will be automatically connected with the imaging apparatus 400 and the printer 495 through wireless communication channels. The images captured by the imaging apparatus 400 can be sent to the computing module 498 in the base station 490 and displayed on the display monitor 494 in real time, while the same images can also be stored in the electronic data storage unit 497, and printed out by the printer 495. The electronic data storage unit 497, which stores all of the patient information and pictures, can be removed from the carrying case 490 and placed in a safe location. When the electronic system of the base station 490 is powered up, the communication module 493 can also automatically connect with local area computer network or internet wirelessly. Such connection enables the data exchanges between the electronic data storage unit 497 and data storages connected with the local area computer network or internet. By pushing down the power on/off switch 499b again, the whole electronic system in the base station 490 can be shut down automatically.

FIG. 4B is a perspective view that schematically illustrates the carrying case 490 comprising an electrical recharging station 482. In various embodiments, the electrical recharging station 482 allows the users to recharge the imaging apparatus 400 during and/or after the imaging session. The electrical recharging station 482 can comprise a plurality of retractable electrical contacts 483. Through the power ports built into the housing of the imaging apparatus 400 and corresponding retractable electrical contacts 483 in the electrical recharging station 482, the battery in the imaging apparatus 400 can be recharged. When the imaging apparatus 400 is plugged into the re-charging station 482, the station 482 can further provide a safe and secure resting station for the imaging apparatus 400 when it is not being used for photographing the patients.

FIG. 4C is a schematic view that illustrates some other embodiments of the base station 480. To enable the convenience of being used in clinical and surgical rooms, the carrying case 490 can be placed on a mobile cart 481. The cart 481 can be built with multiple shelves and wheels in order to store multiple devices and to allow easy maneuvering in tight spaces. The carrying case 490 may be placed on one of the shelves with the eye imaging apparatus 400 stored inside the carrying case 490. The user may take out the entire case 490 from the cart 481 and use the case 490 in other locations, or may use the case 490 for storage in the cart 481. The image computing module 498, the display 494, the keyboard 496, and the printer 495 may also be placed in the carrying case 490 and may be used in the same manner as described in the above paragraphs. When the carrying case 490 is placed on the shelf of the cart 481, a power cord of the case may be connected directly into the electric power supply system of the cart; the battery of the case 490 may be recharged automatically. In some embodiments, the base station may also include a foot switch 485. The foot switch 485 can be configured to communicate with wireless imaging apparatus 400 wirelessly. The user can control the imaging capture process by pushing the foot switch 485. The foot switch 485 can send a command signal wirelessly to the wireless imaging apparatus 400.

FIG. 4D is a block diagram that schematically illustrates a wireless imaging system 500 comprising the wireless imaging apparatus 400 and the base station 490. Unless otherwise noted, reference numerals used in FIG. 4D represent components similar to those illustrated in FIG. 3C, with the reference numerals incremented by 100. The wireless imaging system 500 can comprise the wireless imaging apparatus 300, which can further include a miniature camera 426, a light source 423, a light source driver 435, a modified mobile device 404, a microcontroller 475 and a multi-functional button 450. The modified mobile device 404 can comprise a touch screen 405, an input port 475, and a USB port 479. The user can place the imaging apparatus 400 on an object and preview the image on the touch screen 404. The imaging apparatus can be configured to communicate with a base station 490 wirelessly. The preview of the image can also be shown on a display monitor 494 of the base station 490. The preview of the image can assist the user to perform precise alignment, focus adjustment and light intensity control. After the user finishes the alignment and adjustment, the user can push the multi-functional button 450. The multi-functional button 450 can communicate with the microcontroller 439, and the microcontroller 439 can communicate with the modified mobile device 404 to start the image capturing process. In some embodiments, the base station 490 can comprise a foot switch 485. For example, the foot switch 485 can send a command signal wirelessly to the modified mobile device 404, and the modified mobile device 404 can transmit the command signal to the microcontroller 439 through the audio port 474. The images can be transmitted wirelessly to the base station 490. The images can be printed by a printer 495 in the base station 490. The images can be further transmitted wirelessly to a computing device in a hospital or medical facility, to be evaluated by a physician in real time.

While the present disclosure has been disclosed in example embodiments, those of ordinary skill in the art will recognize and appreciate that many additions, deletions and modifications to the disclosed embodiments and their variations may be implemented without departing from the scope of the disclosure. A wide range of variations to those implementations and embodiments described herein are possible. Components and/or features may be added, removed, rearranged, or combinations thereof. Similarly, method steps may be added, removed, and/or reordered.

Likewise various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Accordingly, reference herein to a singular item includes the possibility that a plurality of the same item may be present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below.

Additionally as used herein, a phrase referring to “at least one of” a list of items refers to any combination of those items, including single members. As an example, “at least one of: a, b, or c” is intended to cover: a, b, c, a-b, a-c, b-c, and a-b-c.

Certain features that are described in this specification in the context of separate embodiments also can be implemented in combination in a single embodiment. Conversely, various features that are described in the context of a single embodiment also can be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be described as occurring in a particular order, this should not be understood as requiring that such operations be performed in the particular order described or in sequential order, or that all described operations be performed, to achieve desirable results. Further, other operations that are not disclosed can be incorporated in the processes that are described herein. For example, one or more additional operations can be performed before, after, simultaneously, or between any of the disclosed operations. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single product or packaged into multiple products. Additionally, other embodiments are within the scope of the following claims. In some cases, the actions recited in the claims can be performed in a different order and still achieve desirable results.

The systems, devices, and methods of the preferred embodiments and variations thereof can be embodied and/or implemented at least in part as a machine configured to receive a computer-readable medium storing computer-readable instructions. The instructions are preferably executed by computer-executable components preferably integrated with the system including the computing device configured with software. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-20. (canceled)

21. A wireless eye imaging apparatus comprising:
a housing comprising a first portion and a second portion;
a light source disposed inside and supported by the first portion, the light source configured to illuminate an eye;
an imaging capturing unit disposed inside the first portion, the imaging capturing unit comprising
a focusing lens,
an actuator configured to change a focus of an optical imaging system, and
an image sensor configured to receive an image of the eye;
a modified mobile device disposed inside and being supported by the second portion, the modified mobile device comprising
a wireless transmitter and a wireless receiver,
a processor configured to control the light source, the actuator and the image sensor,
a display configured to display the image, and
wherein the modified mobile device is adapted from a mobile device, wherein the focusing lens comprises a lens of the mobile device being repositioned to be outside of the modified mobile device, wherein the image sensor comprises an image sensor of the mobile device being repositioned to be outside of the modified mobile device;
a cable disposed inside the housing and operatively connected to the imaging capturing unit and the modified mobile device and having a length between 5 mm and 15 mm; and
a microcontroller disposed inside the housing and operatively connected to the modified mobile device, the light source and the imaging capturing unit, the microcontroller configured to control and synchronize the light source, the actuator and the image sensor.

22. The wireless eye imaging apparatus in claim 21, wherein the modified mobile device comprises a modified smart phone.

23. The wireless eye imaging apparatus in claim 21, further comprising a multi-functional button disposed on an exterior surface of the housing, wherein the multi-functional button comprises electrical switches operatively connected to the light source, the actuator and the imaging sensor.

24. The wireless eye imaging apparatus in claim 23, wherein the multi-functional button is configured to control the light source, the actuator and the imaging sensor.

25. The wireless eye imaging apparatus in claim 23, wherein the multi-functional button is configured to receive a trigger signal in response to a pushing action on the multi-functional button and to send a second signal to the modified mobile device.

26. The wireless eye imaging apparatus in claim 21, wherein at least one of an input port, an output port, a control button, an input signal and an output signal of the modified mobile device is connected to the microcontroller.

27. The wireless eye imaging apparatus in claim 21, wherein at least one of the input port, the output port, the control button, the input signal and the output signal of the modified mobile device is connected to one of the light source and the imaging capturing unit.

28. The wireless eye imaging apparatus in claim 21, wherein the light source comprises a plurality of light emitting elements configured to illuminate different portions of the eye time sequentially, wherein an output signal of the modified mobile device is configured to be connected to the microcontroller to synchronize the plurality of light emitting elements with the imaging sensor.

29. The wireless eye imaging apparatus in claim 21, further comprising an independent driver to drive the light source, wherein the microcontroller is further configured to control the light source.

30. The wireless eye imaging apparatus in claim 21, wherein an audio port of the mobile device is modified to transmit a command signal by encoding the command signal into an audio signal and recovering the command signal by decoding the audio signal.

31. A wireless eye imaging apparatus comprising:
a housing comprising a first portion and a second portion;
a light source disposed inside and supported by the first portion, the light source configured to illuminate an eye;
an imaging capturing unit disposed inside the first portion, the imaging capturing unit comprising
a focusing lens,
an actuator configured to change a focus of an optical imaging system, and
an image sensor configured to receive an image of the eye;
a modified mobile device disposed inside and being supported by the second portion, the modified mobile device comprising
a wireless transmitter and a wireless receiver,
a processor configured to control the light source, the actuator and the image sensor,
a display configured to display the image, and
wherein the modified mobile device is adapted from a mobile device, wherein an audio port of the mobile device is modified to transmit a command signal by encoding the command signal into an audio signal and recovering the command signal by decoding the audio signal; and
a microcontroller disposed inside the housing and operatively connected to the modified mobile device, the light source and the imaging capturing unit, the microcontroller configured to control and synchronize the light source, the actuator and the image sensor.

32. The wireless imaging apparatus in claim 31, wherein an audio input port of the modified mobile device is used to receive a command signal.

33. The wireless imaging apparatus in claim 31, wherein an audio output port of the modified mobile device is used to transmit a command signal.

34. The wireless eye imaging apparatus in claim 31, wherein the modified mobile device comprises a modified smart phone.

35. The wireless eye imaging apparatus in claim 31, further comprising a multi-functional button disposed on an exterior surface of the housing, wherein the multi-functional button comprises electrical switches operatively connected to the light source, the actuator and the imaging sensor.

36. The wireless eye imaging apparatus in claim 35, wherein the multi-functional button is configured to control the light source, the actuator and the imaging sensor.

37. The wireless eye imaging apparatus in claim 31, wherein at least one of an input port, an output port, a control button, an input signal and an output signal of the modified mobile device is connected to the microcontroller.

38. The wireless eye imaging apparatus in claim 31, wherein at least one of the input port, the output port, the control button, the input signal and the output signal of the modified mobile device is connected to one of the light source and the imaging capturing unit.

39. The wireless eye imaging apparatus in claim 31, wherein the light source comprises a plurality of light emitting elements configured to illuminate different portions of the eye time sequentially, wherein an output signal of the modified mobile device is configured to be connected to the microcontroller to synchronize the plurality of light emitting elements with the imaging sensor.

40. The wireless eye imaging apparatus in claim 31, wherein the focusing lens comprises a lens of the mobile device being repositioned to be outside of the modified mobile device, wherein the image sensor comprises an image sensor of the mobile device being repositioned to be outside of the modified mobile device, wherein the wireless eye imaging apparatus further comprises a cable disposed inside the housing and operatively connected to the imaging capturing unit and the modified mobile device and having a length between 5 mm and 15 mm.
